(19) United States
(12) Patent Application Publication
(10) Pub. No.: US 2014/0132747 A1
(43) Pub. Date: May 15, 2014

(54) DIGITAL INTRA-ORAL PANARAMIC ARCH CAMERA

(71) Applicant: Jessica Stephanie Andrews, Middle Village, NY (US)

(72) Inventor: Jessica Stephanie Andrews, Middle Village, NY (US)

(21) Appl. No.: 13/678,510

(22) Filed: Nov. 15, 2012

(51) Int. Cl. A61B 1/24

(52) U.S. Cl. CPC A61B 1/24
USPC 348/66

(57) ABSTRACT

A digital intra-oral panoramic arch camera can provide a single panoramic image easily taken by one person. The intra-oral panoramic arch camera can save clinical time and improve accuracy for treatment planning. The intra-oral panoramic arch camera includes a plurality of image capturing devices and a plurality of lamps, such as light emitting diodes, for providing sufficient lighting for capturing the image. The intra-oral panoramic arch camera can include adjustable positioning arms to adjust the individual cameras to the correct positioning depending on the patient's oral structure.

### BACKGROUND OF THE INVENTION

The present invention relates to dental care accessories and, more particularly, to a digital intro-oral panoramic arch camera.

While trying to capture full arch occlusal photos, intra-oral cameras only capture sections and waste time. In order to take occlusal photos, doctors need expensive cameras and use mirrors and often need assistance doing so. This can use valuable appointment time and does not put the full arch together as one to make accurate diagnosis and treatment planning.

As can be seen, there is a need for an intra-oral camera that can quickly and easily capture a panoramic digital arch photo.

### SUMMARY OF THE INVENTION

In one aspect of the present invention, a dental camera comprises a plurality of camera enclosures disposed about an arc; a plurality of adjustment arms attaching each of the plurality of cameras to adjustment rings, wherein the plurality of adjustment arms are movable between an expanded configuration and a retracted configuration; at least one camera and at least one light disposed in each of the plurality of camera enclosures; a main body attached to at least one of the plurality of camera enclosures; and an action button disposed in the main body, the action button causing the lights to illuminate and a picture to be taken on each of the cameras.

In another aspect of the present invention, an intra-oral panoramic arch camera comprises a plurality of camera enclosures disposed about an arc; a plurality of adjustment arms attaching each of the plurality of cameras to adjustment rings, wherein the plurality of adjustment arms are movable between an expanded configuration and a retracted configuration; at least one camera and at least one light disposed in each of the plurality of camera enclosures; a main body attached to at least one of the plurality of camera enclosures; an action button disposed in the main body, the action button causing the lights to illuminate and a picture to be taken on each of the cameras; a power button disposed in the main body; a finger rest disposed on one side of the camera body; and a port formed in the main body, the port permitting the dental camera to send pictures and receive a power charge.

In a further aspect of the present invention a method for obtaining an intra-oral panoramic arch image comprises retracting a plurality of camera enclosures of a dental camera, the plurality of camera enclosures disposed on adjustment arms about an arc, the camera enclosures having at least one camera and at least one lamp disposed therein; holding a main body of the dental camera; inserting the camera enclosures into a mouth of a patient; adjusting a length of the camera arms to position the camera enclosures in a desired position; and depressing an action button to take a picture with the cameras.

These and other features, aspects and advantages of the present invention will become better understood with reference to the following drawings, description and claims.

### BRIEF DESCRIPTION OF THE DRAWINGS

FIG. 1 is a perspective top view of an intra-oral panoramic arch camera according to an exemplary embodiment of the present invention;

FIG. 2 is a partially cut-away side view of the intra-oral panoramic arch camera of FIG. 1, in use in a patient;

FIG. 3 is a rear perspective view of the intra-oral panoramic arch camera of FIG. 1;

FIG. 4 is a top view of the intra-oral panoramic arch camera of FIG. 1 illustrated in an expanded configuration;

FIG. 5 is a top view of the intra-oral panoramic arch camera of FIG. 1 illustrated in a contracted configuration;

FIG. 6 is a front perspective view of the intra-oral panoramic arch camera of FIG. 1 illustrated in a contracted configuration;

FIG. 7 is a rear perspective view of the intra-oral panoramic arch camera of FIG. 1 illustrated in the contracted configuration;

FIG. 8 is a bottom perspective view of the intra-oral panoramic arch camera of FIG. 1 illustrated in an expanded configuration; and

FIG. 9 is a side view of the intra-oral panoramic arch camera of Figure

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a digital intra-oral panoramic arch camera that can provide a single panoramic image easily taken by one person. The intra-oral panoramic arch camera can save clinical time and improve accuracy for treatment planning. The intra-oral panoramic arch camera includes a plurality of image capturing devices and a plurality of lamps, such as light emitting diodes, for providing sufficient lighting for capturing the image. The intra-oral panoramic arch camera can include adjustable positioning arms to adjust the individual cameras to the correct positioning depending on the patient's oral structure.

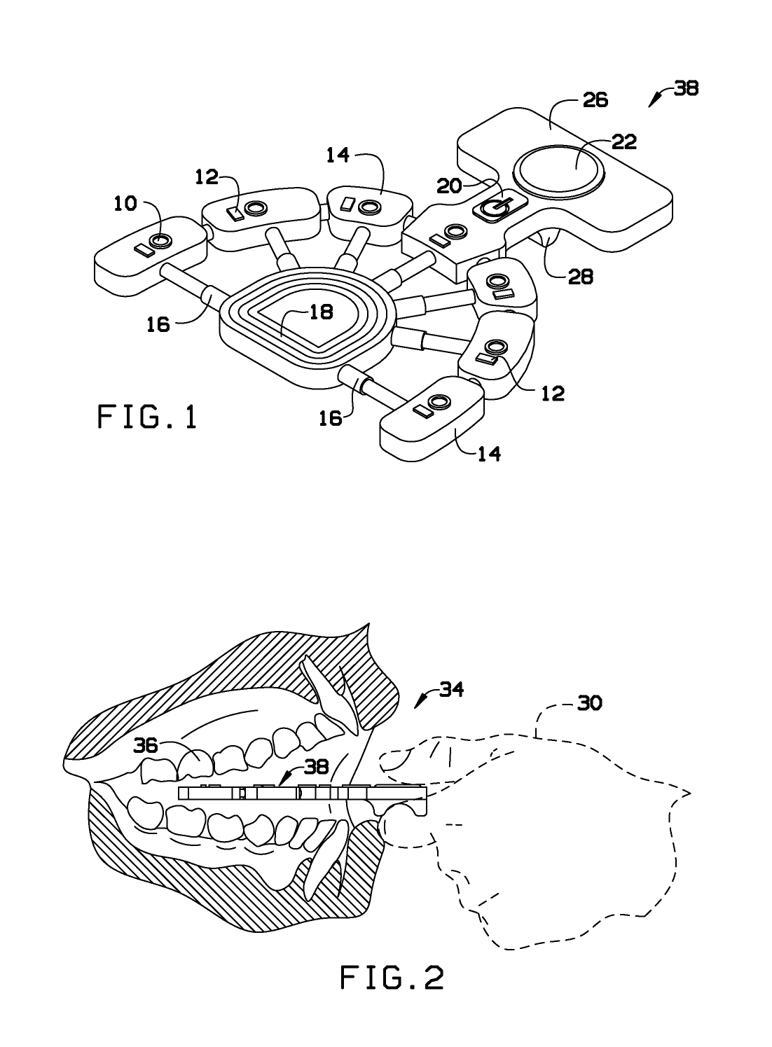

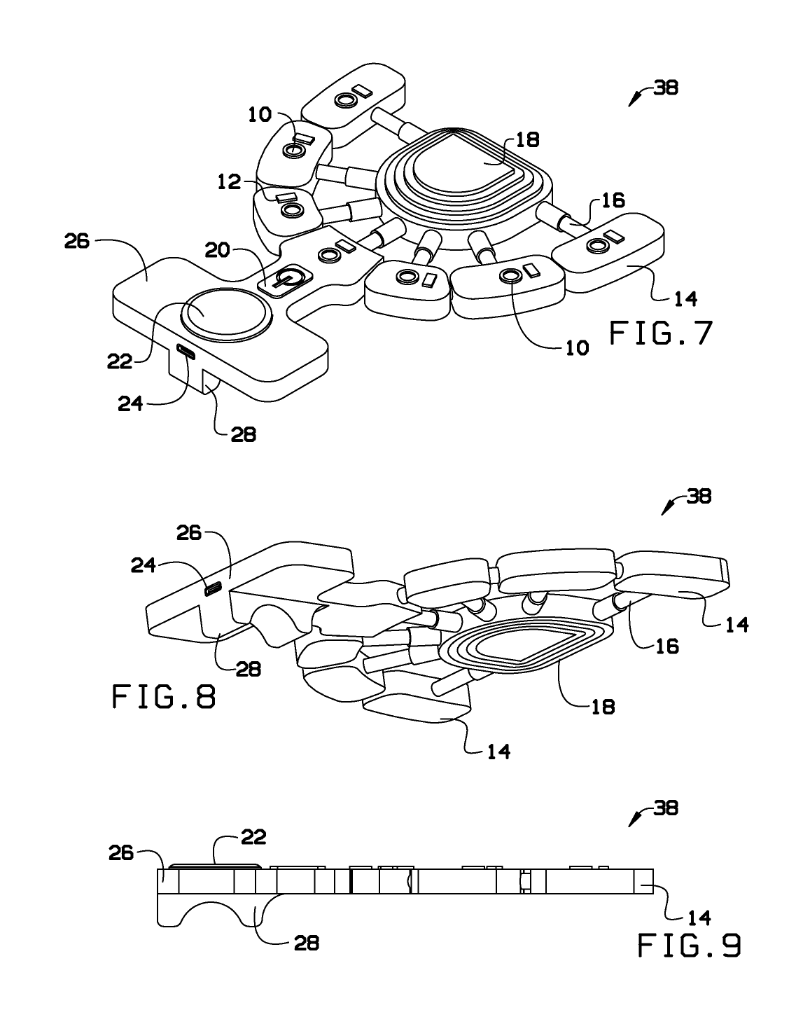

Referring now to FIGS. 1 through 9, an intra-oral full arch adjustable dental camera 38 can include a plurality of cameras 10 and a plurality of lights, such as light emitting diodes (LEDs) 12 disposed on a plurality of camera enclosures 14. Each of the camera enclosures can connect to adjustment rings 18 via adjustment arms 16. Typically, as shown in the Figures, seven camera enclosures 14 can be adjustably disposed about the adjustment rings 18, each on their own adjustment arm 16. While the Figures show one camera 10 and one LED 12 on each camera enclosure 14, other numbers of cameras 10 and LEDs 12 can be disposed on each camera enclosure 14 and the number of cameras 10 and LEDs 12 on each camera enclosure 14 can be the same or different.

Typically the camera enclosures 14 form a semi-circular arc about the adjustment rings 18. However, each adjustment arm 16 can be independently adjusted to form imperfect semi-circular shapes depending on the shape of a patient's mouth 34 and position of their teeth 36.

A central one of the camera enclosures 14 can connect to a main body 26. The main body 26 can include a finger rest 28 to assist a user 30 in handling the dental camera 38.

The main body 26 can include a power button 20 for turning on and off the dental camera 38. The main body 26 can further include an action button 20 for taking a picture. The main body 26 can further include a port 24. The port 24 can be used for charging a battery (not shown) of the dental camera 38 and form transferring pictures taken by the cameras 10 to a computing device (not shown). In some embodiments, the port 24 can be designed to connect to a computing device, during use of the dental camera 38, to allow the user to visualize the view from the cameras 10 prior to taking the picture. This design could be similar to a conventional digital camera that has a view screen, where the user can visualize what they are going to take a picture of prior to taking the picture. In some embodiments, a cable can extend from where the port 24 is located, where this cable can be hard-wired into the dental camera 38 and connected to a computing device, for example.

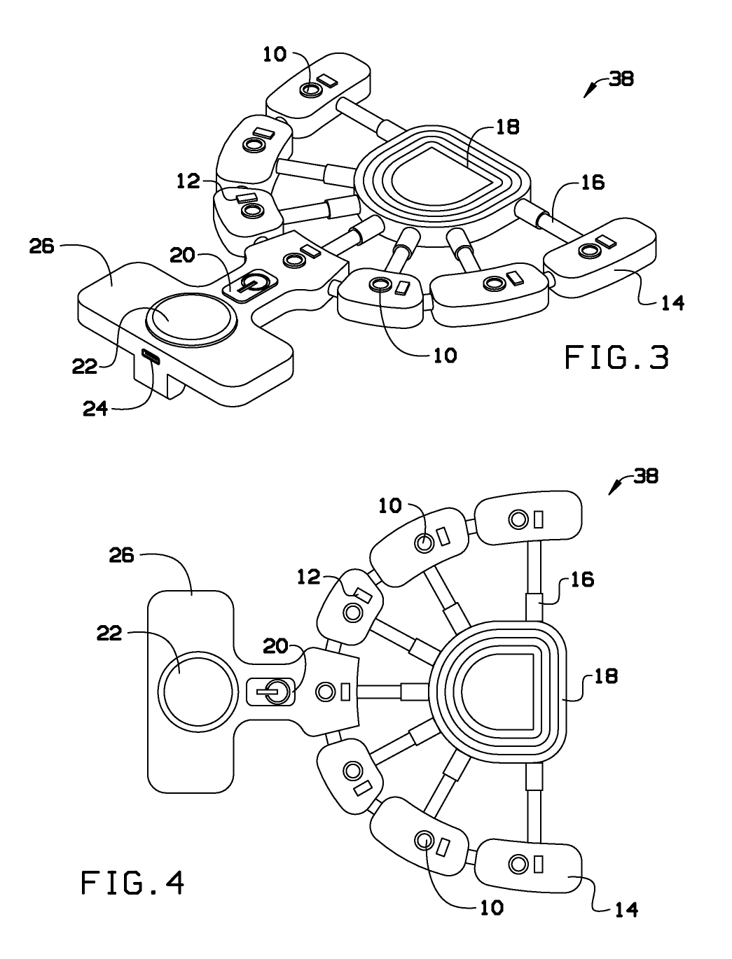

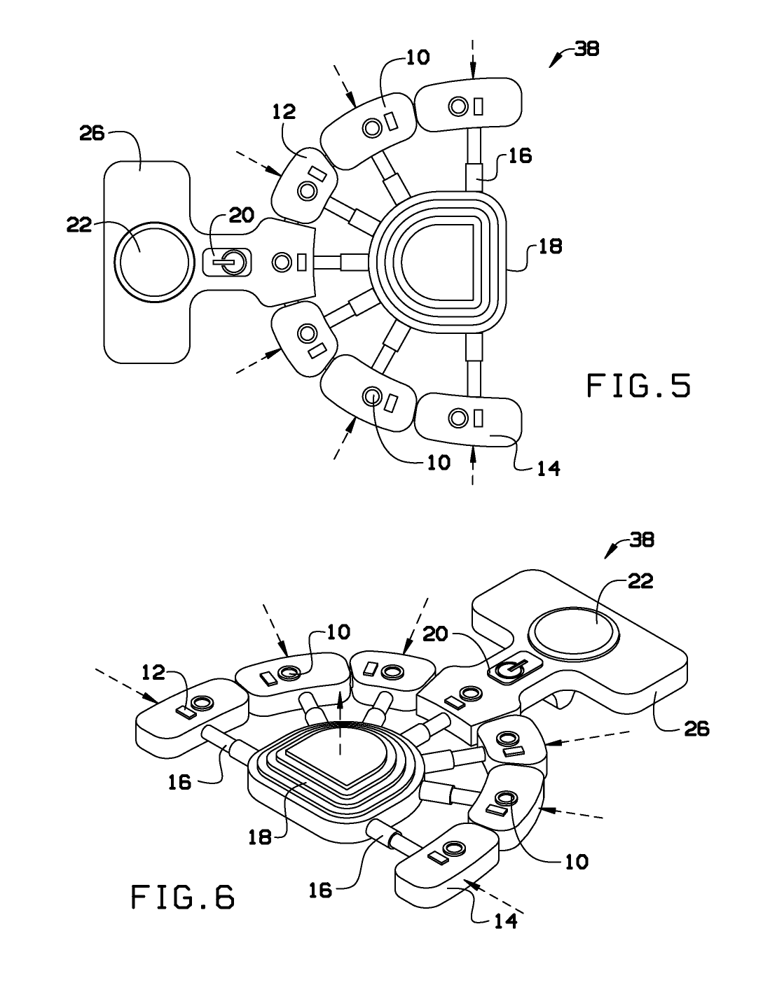

To use the dental camera 38 of the present invention, the user 30 can insert the dental camera 38 into the patient's mouth 34 in a retracted configuration (see FIGS. 5-7, for example). A disposable barrier for infection control can be used to cover the dental camera 38 prior to insertion into the patient's mouth 34. The adjustment rings 18 can then be used to adjust the position and length of the adjustment arms 16 to position each of the camera enclosures in a desired expanded configuration (see FIGS. 3 and 4, for example). Once in position, the user can depress the action button 22 to take a picture, illuminated by the LEDs 12.

In some embodiments of the present invention, the camera enclosures can be made to not only extend and retract to and from the adjustment rings 18, but can be designed to angle, in some embodiments, to angle to a vertical position, to capture buccal and lingual surfaces of teeth.

The present invention can also include a software application that can obtain the pictures from each of the cameras 10 of the dental camera 38. The software application can then process the pictures into a single intra-oral panoramic arch image.

It should be understood, of course, that the foregoing relates to exemplary embodiments of the invention and that modifications may be made without departing from the spirit and scope of the invention as set forth in the following claims.

What is claimed is:

1. A dental camera comprising:
a plurality of camera enclosures disposed about an arc;
a plurality of adjustment arms attaching each of the plurality of cameras to adjustment rings, wherein the plurality of adjustment arms are movable between an expanded configuration and a retracted configuration;
at least one camera and at least one light disposed in each of the plurality of camera enclosures;
a main body attached to at least one of the plurality of camera enclosures; and
an action button disposed in the main body, the action button causing the lights to illuminate and a picture to be taken on each of the cameras.

2. The dental camera of claim 1, wherein one camera and one light are disposed in each of the plurality of camera enclosures.

3. The dental camera of claim 1, wherein the plurality of camera enclosures are seven camera enclosures.

4. The dental camera of claim 1, further comprising a power button disposed in the main body.

5. The dental camera of claim 1, further comprising a finger rest disposed on one side of the camera body.

6. The dental camera of claim 1, further comprising a port formed in the main body, the port permitting the dental camera to send pictures and receive a power charge.

7. The dental camera of claim 1, wherein the light is a light emitting diode.

8. An intra-oral panoramic arch camera comprising:
a plurality of camera enclosures disposed about an arc;
a plurality of adjustment arms attaching each of the plurality of cameras to adjustment rings, wherein the plurality of adjustment arms are movable between an expanded configuration and a retracted configuration;
at least one camera and at least one light disposed in each of the plurality of camera enclosures;
a main body attached to at least one of the plurality of camera enclosures;
an action button disposed in the main body, the action button causing the lights to illuminate and a picture to be taken on each of the cameras;
a power button disposed in the main body;
a finger rest disposed on one side of the camera body; and
a port formed in the main body, the port permitting the dental camera to send pictures and receive a power charge.

9. The dental camera of claim 8, wherein one camera and one light are disposed in each of the plurality of camera enclosures.

10. The dental camera of claim 9, wherein the plurality of camera enclosures are seven camera enclosures.

11. A method for obtaining an intra-oral panoramic arch image, the method comprising:
retracting a plurality of camera enclosures of a dental camera, the plurality of camera enclosures disposed on adjustment arms about an arc, the camera enclosures having at least one camera and at least one lamp disposed therein;
holding a main body of the dental camera;
inserting the camera enclosures into a mouth of a patient;
adjusting a length of the camera arms to position the camera enclosures in a desired position; and
depressing an action button to take a picture with the cameras.
